(19) United States
(12) Patent Application Publication
(10) Pub. No.: US 2023/0135548 A1
(43) Pub. Date: May 4, 2023

(54) INFORMATION PROCESSING APPARATUS, INFORMATION PROCESSING METHOD, AND INFORMATION PROCESSING PROGRAM

(71) Applicant: FUJIFILM Corporation, Tokyo (JP)

(72) Inventors: Toru NISHINO, Tokyo (JP); Tomoko OHKUMA, Tokyo (JP); Tomoki TANIGUCHI, Tokyo (JP)

(73) Assignee: FUJIFILM Corporation, Tokyo (JP)

(21) Appl. No.: 18/050,044

(22) Filed: Oct. 26, 2022

(30) Foreign Application Priority Data
Oct. 29, 2021 (JP) 2021-178211

(51) Int. Cl. G16H 15/00; G06F 40/56; G06T 7/00; G06T 7/11

(52) U.S. Cl. CPC G16H 15/00; G06F 40/56; G06T 7/0012; G06T 7/11; G06T 2207/30064

(57) ABSTRACT

An information processing apparatus comprising at least one processor, wherein the at least one processor is configured to: acquire a plurality of pieces of element information related to an image; generate a plan which defines a description order of elements corresponding to the plurality of pieces of element information in a sentence in which the elements are described; and generate the sentence based on the plan.

### CROSS-REFERENCE TO RELATED APPLICATIONS

The present application claims priority under 35 U.S.C. § 119 to Japanese Patent Application No. 2021-178211, filed on Oct. 29, 2021. The above application is hereby expressly incorporated by reference, in its entirety, into the present application.

### BACKGROUND

### Technical Field

The present disclosure relates to an information processing apparatus, an information processing method, and an information processing program.

### Related Art

In the related art, image diagnosis is performed using medical images obtained by imaging apparatuses such as computed tomography (CT) apparatuses and magnetic resonance imaging (MRI) apparatuses. In addition, image diagnosis is made by analyzing medical images via computer aided detection/diagnosis (CAD) using a discriminator in which learning is performed by deep learning or the like, and detecting and/or diagnosing regions of interest including structures, lesions, and the like included in the medical images. The medical images and analysis results via CAD are transmitted to a terminal of a healthcare professional such as a radiologist who interprets the medical images. The healthcare professional such as a radiologist interprets the medical image by referring to the medical image and analysis result using his or her own terminal and creates an interpretation report.

In addition, various methods have been proposed to support the creation of medical documents such as interpretation reports in order to reduce the burden of the interpretation work of a radiologist. For example, JP2019-153250A discloses a technique for creating a medical document such as an interpretation report based on a keyword input by a radiologist and an analysis result of a medical image. In the technique described in JP2019-153250A, a sentence to be included in the interpretation report is created by using a recurrent neural network trained to generate a sentence from input characters. Further, for example, JP2008-257579A discloses a technique for creating a fixed form associated with each type of annotation in advance for a medical image with an annotation as comments on findings of the medical image.

In recent years, as the performance of imaging apparatuses has improved, the amount of information on analysis results obtained from medical images has tended to increase, and therefore the amount of sentences described in medical documents such as interpretation reports has also tended to increase. In order to make it easier to read a large amount of sentences, in medical documents, rules such as agreements within medical institutions and user's preferences regarding a description order of analysis results obtained from medical images may be determined.

For example, in a case of describing an abnormal shadow in a medical image, it may be desirable to describe the overall properties of the abnormal shadow first, and the marginal portion and internal properties later. Further, for example, it may be desired to describe the malignant findings first and the benign findings later. Further, for example, in a case of describing the comparison result with the past medical image, it may be desired to describe the changed portion first and the unchanged portion later.

However, in the related art, in a case of trying to generate a sentence including a large amount of complicated information, there are cases where the sentences are not in the desired order of description, the information is omitted, or the sentences become redundant. Therefore, there is a demand for a technique capable of generating sentences with an appropriate description order and degree of coverage of information for sentences described in medical documents and the like.

### SUMMARY

The present disclosure provides an information processing apparatus, an information processing method, and an information processing program capable of supporting creation of medical documents.

According to a first aspect of the present disclosure, there is provided an information processing apparatus comprising at least one processor, in which the processor is configured to acquire a plurality of pieces of element information related to an image, generate a plan which defines a description order of elements corresponding to the plurality of pieces of element information in a sentence in which the elements are described, and generate the sentence based on the plan.

In the first aspect, the processor may be configured to divide the plurality of pieces of element information into groups, and generate the plan which defines the description order for each group.

In the first aspect, the processor may be configured to receive a designation of at least one of a plurality of different rules predetermined for a method of dividing the plurality of pieces of element information into the groups, and divide the plurality of pieces of element information into groups according to the designated rule.

In the first aspect, the processor may be configured to acquire a plurality of pieces of element information related to each of a plurality of regions of interest included in the image, and divide the plurality of pieces of element information into a plurality of groups corresponding to each of the plurality of regions of interest.

In the first aspect, the plurality of pieces of element information may be related to each of a plurality of the images, and the processor may be configured to generate a sentence in which elements corresponding to the plurality of pieces of element information related to each of the plurality of images are collectively described.

In the first aspect, the processor may be configured to acquire element information indicating an imaging point in time of the image, and generate the plan which defines the description order of an element corresponding to related element information based on the imaging point in time indicated by the element information.

In the first aspect, an importance may be predetermined for each piece of element information, and the processor may be configured to generate the plan which defines that an element corresponding to element information whose importance is lower than a predetermined threshold value among the plurality of pieces of element information is not described in the sentence.

In the first aspect, an importance may be predetermined for each piece of element information, and the processor may be configured to generate the plan which defines that an element corresponding to element information having a relatively low importance among the plurality of pieces of element information is not described in the sentence.

In the first aspect, the processor may be configured to receive a designation of a degree of conciseness of the sentence, and change the number of elements not to be described in the sentence depending on the degree of conciseness.

In the first aspect, the processor may be configured to generate the plan using a first trained model that has been trained in advance so that an input is the element information and an output is the plan, and generate the sentence using a second trained model that has been trained in advance so that an input is the plan and an output is the sentence.

In the first aspect, the first trained model may be trained using a set of the element information corresponding to the element included in the sentence generated in a past and the plan which defines the description order of the elements in the sentence as training data.

In the first aspect, the processor may be configured to generate a plurality of different candidates of the plan for the plurality of pieces of element information, generate the sentence for each candidate of the plan, evaluate each sentence, and determine a candidate of the plan to be employed based on a result of the evaluation.

In the first aspect, the processor may be configured to perform the evaluation based on at least one of the description order or a degree of coverage of the elements included in the generated sentence in the sentence.

In the first aspect, the processor may be configured to generate a plurality of different candidates of the plan for the plurality of pieces of element information, and receive a designation of a candidate of the plan to be employed from among the plurality of different candidates of the plan.

In the first aspect, the processor may be configured to acquire the image, and generate the element information based on the acquired image.

In the first aspect, the information processing apparatus may further comprise an input unit, and the processor may be configured to generate the element information based on information input via the input unit.

In the first aspect, the element information may be information indicating at least one of a name, a property, a measured value, or a position related to a region of interest included in the image, or an imaging method, an imaging condition, or an imaging date and time related to imaging of the image.

In the first aspect, the image may be a medical image, the element information may be information indicating at least one of a name, a property, a position, or an estimated disease name related to a region of interest included in the medical image, or an imaging method, an imaging condition, or an imaging date and time related to imaging of the medical image, and the region of interest may be at least one of a region of a structure included in the medical image or a region of an abnormal shadow included in the medical image.

According to a second aspect of the present disclosure, there is provided an information processing method comprising: acquiring a plurality of pieces of element information related to an image; generating a plan which defines a description order of elements corresponding to the plurality of pieces of element information in a sentence in which the elements are described; and generating the sentence based on the plan.

According to a third aspect of the present disclosure, there is provided an information processing program causing a computer to execute: acquiring a plurality of pieces of element information related to an image; generating a plan which defines a description order of elements corresponding to the plurality of pieces of element information in a sentence in which the elements are described; and generating the sentence based on the plan.

The information processing apparatus, the information processing method, and the information processing program according to the aspects of the present disclosure can support the creation of medical documents.

### BRIEF DESCRIPTION OF THE DRAWINGS

FIG. 1 is a diagram showing an example of a schematic configuration of an information processing system.

FIG. 2 is a block diagram showing an example of a hardware configuration of an information processing apparatus.

FIG. 3 is a block diagram showing an example of a functional configuration of an information processing apparatus according to a first embodiment.

FIG. 4 is a diagram showing an example of a medical image.

FIG. 5 is a diagram showing an example of element information.

FIG. 6 is a diagram showing an example of a sentence.

FIG. 7 is a diagram for describing a process according to the first embodiment.

FIG. 8 is a diagram showing an example of a plan.

FIG. 9 is a diagram showing an example of a screen displayed on a display.

FIG. 10 is a flowchart showing an example of first information processing.

FIG. 11 is a block diagram showing an example of a functional configuration of an information processing apparatus according to a second embodiment.

FIG. 12 is a diagram for describing a process according to the second embodiment.

FIG. 13 is a diagram showing an example of a plan candidate.

FIG. 14 is a diagram showing an example of a plan candidate.

FIG. 15 is a diagram showing an example of a plan candidate.

FIG. 16 is a flowchart showing an example of second information processing.

FIG. 17 is a diagram for describing a process according to a method of the related art.

FIG. 18 is a diagram showing an example of a sentence.

FIG. 19 is an evaluation result of the methods according to the first and second embodiments and the method of the related art.

FIG. 20 is a diagram showing an example of a plan.

FIG. 21 is a diagram showing an example of a plan.

FIG. 22 is a diagram showing an example of a plan.

FIG. 23 is a diagram showing an example of a plan.

FIG. 24 is a diagram showing an example of a screen displayed on a display.

FIG. 25 is a diagram showing an example of a screen displayed on a display.

### DETAILED DESCRIPTION

Each embodiment of the present disclosure will be described below with reference to the drawings.

### First Embodiment

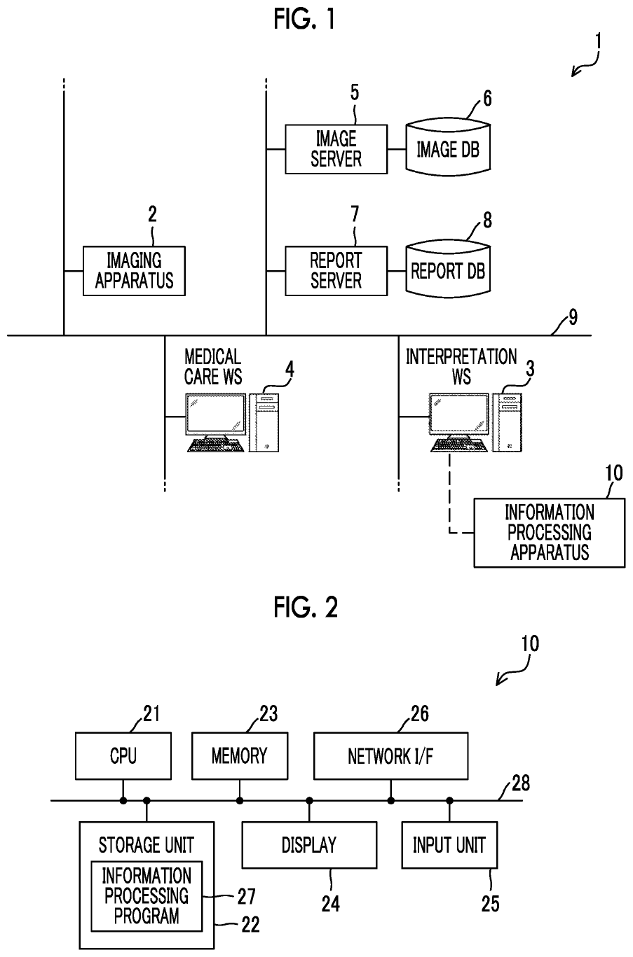

First, a configuration of an information processing system 1 to which an information processing apparatus of the present disclosure is applied will be described. FIG. 1 is a diagram showing a schematic configuration of the information processing system 1. The information processing system 1 shown in FIG. 1 performs imaging of an examination target part of a subject and storing of a medical image acquired by the imaging based on an examination order from a doctor in a medical department using a known ordering system. In addition, the information processing system performs an interpretation work of a medical image and creation of an interpretation report by a radiologist and viewing of the interpretation report by a doctor of a medical department that is a request source.

As shown in FIG. 1, the information processing system 1 includes an imaging apparatus 2, an interpretation work station (WS) 3 that is an interpretation terminal, a medical care WS 4, an image server 5, an image database (DB) 6, a report server 7, and a report DB 8. The imaging apparatus 2, the interpretation WS 3, the medical care WS 4, the image server 5, the image DB 6, the report server 7, and the report DB 8 are connected to each other via a wired or wireless network 9 in a communicable state.

Each apparatus is a computer on which an application program for causing each apparatus to function as a component of the information processing system 1 is installed. The application program may be recorded on, for example, a recording medium, such as a digital versatile disc (DVD) or a compact disc read only memory (CD-ROM), and distributed, and be installed on the computer from the recording medium. In addition, the application program may be stored in, for example, a storage apparatus of a server computer connected to the network 9 or in a network storage in a state in which it can be accessed from the outside, and be downloaded and installed on the computer in response to a request.

The imaging apparatus 2 is an apparatus (modality) that generates a medical image showing a diagnosis target part of the subject by imaging the diagnosis target part. Specifically, examples of the imaging apparatus include a simple X-ray imaging apparatus, a CT apparatus, an MRI apparatus, a positron emission tomography (PET) apparatus, and the like. The medical image generated by the imaging apparatus 2 is transmitted to the image server 5 and is saved in the image DB 6.

The interpretation WS 3 is a computer used by, for example, a healthcare professional such as a radiologist of a radiology department to interpret a medical image and to create an interpretation report, and encompasses an information processing apparatus 10 according to the present embodiment. In the interpretation WS 3, a viewing request for a medical image to the image server 5, various image processing for the medical image received from the image server 5, display of the medical image, and input reception of a sentence regarding the medical image are performed. In the interpretation WS 3, an analysis process for medical images, support for creating an interpretation report based on the analysis result, a registration request and a viewing request for the interpretation report to the report server 7, and display of the interpretation report received from the report server 7 are performed. The above processes are performed by the interpretation WS 3 executing software programs for respective processes.

The medical care WS 4 is a computer used by, for example, a healthcare professional such as a doctor in a medical department to observe a medical image in detail, view an interpretation report, create an electronic medical record, and the like, and is configured to include a processing apparatus, a display apparatus such as a display, and an input apparatus such as a keyboard and a mouse. In the medical care WS 4, a viewing request for the medical image to the image server 5, display of the medical image received from the image server 5, a viewing request for the interpretation report to the report server 7, and display of the interpretation report received from the report server 7 are performed. The above processes are performed by the medical care WS 4 executing software programs for respective processes.

The image server 5 is a general-purpose computer on which a software program that provides a function of a database management system (DBMS) is installed. The image server 5 is connected to the image DB 6. The connection form between the image server 5 and the image DB 6 is not particularly limited, and may be a form connected by a data bus, or a form connected to each other via a network such as a network attached storage (NAS) and a storage area network (SAN).

The image DB 6 is realized by, for example, a storage medium such as a hard disk drive (HDD), a solid state drive (SSD), and a flash memory. In the image DB 6, the medical image acquired by the imaging apparatus 2 and accessory information attached to the medical image are registered in association with each other.

The accessory information may include, for example, identification information such as an image identification (ID) for identifying a medical image, a tomographic ID assigned to each tomographic image included in the medical image, a subject ID for identifying a subject, and an examination ID for identifying an examination. In addition, the accessory information may include, for example, information related to imaging such as a imaging method, an imaging condition, and an imaging date and time related to imaging of a medical image. The “imaging method” and “imaging condition” are, for example, a type of the imaging apparatus 2, an imaging part, an imaging protocol, an imaging sequence, an imaging method, the presence or absence of use of a contrast medium, and the like. In addition, the accessory information may include information related to the subject such as the name, age, and gender of the subject.

In a case where the image server 5 receives a request to register a medical image from the imaging apparatus 2, the image server 5 prepares the medical image in a format for a database and registers the medical image in the image DB 6. In addition, in a case where the viewing request from the interpretation WS 3 and the medical care WS 4 is received, the image server 5 searches for a medical image registered in the image DB 6 and transmits the searched for medical image to the interpretation WS 3 and to the medical care WS 4 that are viewing request sources.

The report server 7 is a general-purpose computer on which a software program that provides a function of a database management system is installed. The report server 7 is connected to the report DB 8. The connection form between the report server 7 and the report DB 8 is not particularly limited, and may be a form connected by a data bus or a form connected via a network such as a NAS and a SAN.

The report DB 8 is realized by, for example, a storage medium such as an HDD, an SSD, and a flash memory. In the report DB 8, an interpretation report created in the interpretation WS 3 is registered.

Further, in a case where the report server 7 receives a request to register the interpretation report from the interpretation WS 3, the report server 7 prepares the interpretation report in a format for a database and registers the interpretation report in the report DB 8. Further, in a case where the report server 7 receives the viewing request for the interpretation report from the interpretation WS 3 and the medical care WS 4, the report server 7 searches for the interpretation report registered in the report DB 8, and transmits the searched for interpretation report to the interpretation WS 3 and to the medical care WS 4 that are viewing request sources.

The network 9 is, for example, a network such as a local area network (LAN) and a wide area network (WAN). The imaging apparatus 2, the interpretation WS 3, the medical care WS 4, the image server 5, the image DB 6, the report server 7, and the report DB 8 included in the information processing system 1 may be disposed in the same medical institution, or may be disposed in different medical institutions or the like. Further, the number of each apparatus of the imaging apparatus 2, the interpretation WS 3, the medical care WS 4, the image server 5, the image DB 6, the report server 7, and the report DB 8 is not limited to the number shown in FIG. 1, and each apparatus may be composed of a plurality of apparatuses having the same functions.

Next, the information processing apparatus 10 according to the present embodiment will be described. The information processing apparatus 10 has a function of supporting the creation of a medical document such as an interpretation report based on a medical image captured by the imaging apparatus 2. As described above, the information processing apparatus 10 is encompassed in the interpretation WS 3.

First, with reference to FIG. 2, an example of a hardware configuration of the information processing apparatus 10 according to the present embodiment will be described. As shown in FIG. 2, the information processing apparatus 10 includes a central processing unit (CPU) 21, a non-volatile storage unit 22, and a memory 23 as a temporary storage area. Further, the information processing apparatus 10 includes a display 24 such as a liquid crystal display, an input unit 25 such as a keyboard and a mouse, and a network interface (I/F) 26. The network I/F 26 is connected to the network 9 and performs wired or wireless communication. The CPU 21, the storage unit 22, the memory 23, the display 24, the input unit 25, and the network I/F 26 are connected to each other via a bus 28 such as a system bus and a control bus so that various types of information can be exchanged.

The storage unit 22 is realized by, for example, a storage medium such as an HDD, an SSD, and a flash memory. An information processing program 27 in the information processing apparatus 10 is stored in the storage unit 22. The CPU 21 reads out the information processing program 27 from the storage unit 22, loads the read-out program into the memory 23, and executes the loaded information processing program 27. The CPU 21 is an example of a processor of the present disclosure. As the information processing apparatus 10, for example, a personal computer, a server computer, a smartphone, a tablet terminal, a wearable terminal, or the like can be appropriately applied.

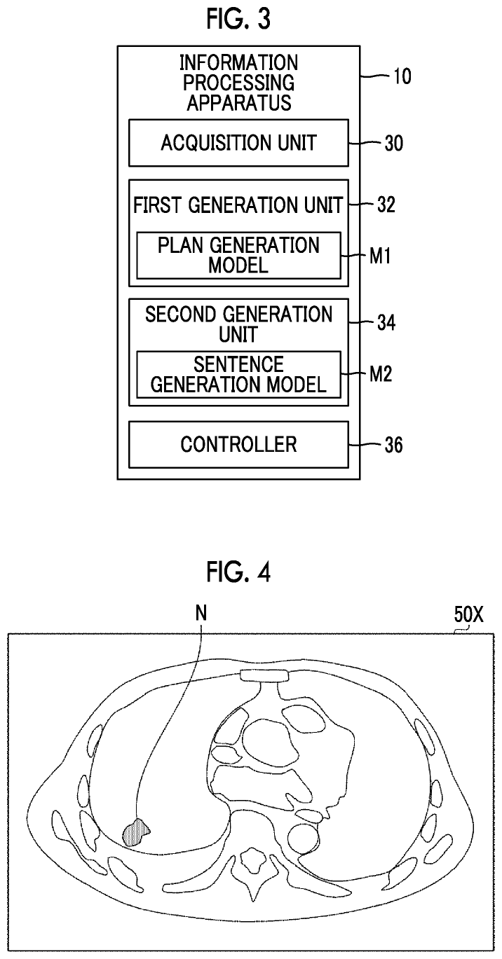

Next, with reference to FIG. 3, an example of a functional configuration of the information processing apparatus 10 according to the present embodiment will be described. As shown in FIG. 3, the information processing apparatus 10 includes an acquisition unit 30, a first generation unit 32, a second generation unit 34, and a controller 36. The first generation unit 32 may include a plan generation model M1, and the second generation unit 34 may include a sentence generation model M2 (details will be described later). In a case where the CPU 21 executes the information processing program 27, the CPU 21 functions as the acquisition unit 30, the first generation unit 32, the second generation unit 34, and the controller 36.

The acquisition unit 30 acquires a medical image to be created as an interpretation report from the image server 5. FIG. 4 shows a medical image 50X obtained by capturing lungs with a CT apparatus as an example of a medical image. The medical image 50X includes an abnormal shadow N indicating a nodule. Hereinafter, at least one of the region of the structure (for example, organs such as lungs and trachea, organum, and tissues) included in the medical image or the region of the abnormal shadow (for example, the shadow due to a lesion such as a nodule) included in the medical image is called a region of interest. Note that one medical image may include a plurality of regions of interest. The medical image is an example of an image of the present disclosure.

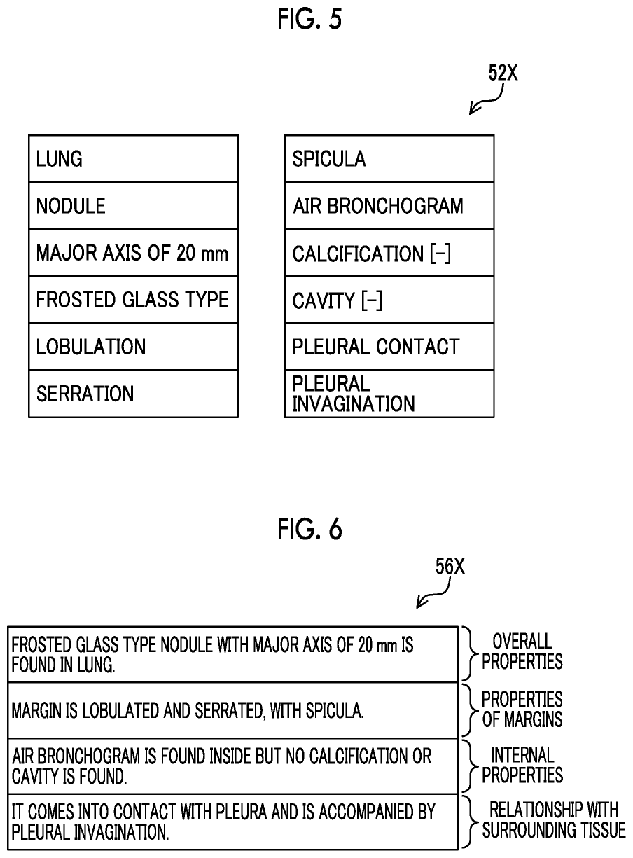

In addition, the acquisition unit 30 acquires a plurality of pieces of element information related to the medical image acquired from the image server 5. FIG. 5 shows, as an example of the element information, a plurality of pieces of element information 52X related to the abnormal shadow N included in the medical image 50X (see FIG. 4). As shown in FIG. 5, the element information may be, for example, information indicating at least one element such as a name (type), a property, a measured value, a position, and an estimated disease name (including a negative or positive evaluation result) related to a region of interest included in a medical image.

Examples of names (types) include the names of structures such as “lung”, “trachea”, and “pleura”, and the names of abnormal shadows such as “nodule”, “cavity”, and “calcification”. The property mainly mean the characteristics of abnormal shadows. For example, in the case of a nodule, findings indicating absorption values such as “solid type” and “frosted glass type”, margin shapes such as “clear/unclear”, “smooth/irregular”, “spicula”, “lobulation”, and “serration”, and an overall shape such as “round shape” and “irregular shape” can be mentioned. Further, for example, findings qualitatively indicating the size and amount of abnormal shadows (“large/small”, “single/multiple”, and the like), and findings regarding the presence or absence of contrast enhancement, washout, and the like can be mentioned.

Examples of the measured value include a value that can be quantitatively measured from a medical image, and examples thereof include a major axis, a CT value whose unit is HU, the number of regions of interest in a case where there are a plurality of regions of interest, and a distance between regions of interest. The position means a position in a medical image regarding a region of interest or a positional relationship with another region of interest, and examples thereof include “inside”, “margin”, and “periphery”. The estimated disease name is an evaluation result estimated by the acquisition unit 30 based on the abnormal shadow, and, for example, the disease name such as “cancer” and “inflammation” and the evaluation result such as “negative/positive” regarding each property, and the like can be mentioned. In FIG. 5, [−] is attached to the property evaluated as negative, and nothing is attached to the property evaluated as positive.

For example, the acquisition unit 30 may generate the above-mentioned element information based on the acquired medical image by using CAD. Specifically, the acquisition unit 30 extracts the region of interest included in the medical image. For the extraction of the region of interest, for example, a trained model such as convolutional neural network (CNN), which has been trained in advance so that the input is a medical image and the output is the region of interest extracted from the medical image, may be used. Further, the acquisition unit 30 may extract a region in the medical image designated by the user via the input unit 25 as a region of interest.

Thereafter, the acquisition unit 30 generates element information related to the region of interest extracted from the medical image. For the generation of the element information by the acquisition unit 30, for example, a trained model such as CNN, which has been trained in advance so that the input is the region of interest in the medical image and the output is the element information related to the region of interest, may be used.

Further, for example, the acquisition unit 30 may generate element information based on the information input via the input unit 25. Specifically, the acquisition unit 30 may generate element information based on the keywords input by the user via the input unit 25. Further, for example, the acquisition unit 30 may present a candidate for element information on the display 24 and receive the designation of the element information by the user.

Further, as described above, each medical image is attached by accessory information including information related to imaging at the time of being registered in the image DB 6. Therefore, for example, the acquisition unit 30 may generate, as element information, information indicating at least one of an imaging method, an imaging condition, or an imaging date and time related to the imaging of the medical image based on the accessory information attached to the medical image acquired from the image server 5.

Further, for example, the acquisition unit 30 may acquire element information generated in advance by an external device having a function of generating element information based on a medical image via CAD as described above from the external device. Further, for example, the acquisition unit 30 may acquire information included in an examination order and an electronic medical record, information indicating various test results such as a blood test and an infectious disease test, information indicating the result of a health diagnosis, and the like from the external device such as the medical care WS 4, and generate the acquired information as element information as appropriate.

Incidentally, in medical documents such as interpretation reports, in order to make it easier for readers to understand the content of the sentence, rules such as an agreement within a medical institution and a user's preference regarding a description order of each element in a sentence in which elements corresponding to a plurality of pieces of element information related to a medical image are described may be determined. For example, in a case of describing an abnormal shadow such as a nodule, it may be desirable to describe the findings such as position, size, and overall shape first, and detailed findings of the marginal portion and the inside later. Further, for example, it may be desired to describe the malignant findings first and the benign findings later. Further, for example, in a case of describing the comparison result of past and present medical images of the same subject as the imaging target, it may be desired to describe the changed portion first and the unchanged portion later.

FIG. 6 shows an example of a sentence 56X in which elements corresponding to the plurality of pieces of element information 52X (see FIG. 5) related to regarding the abnormal shadow N (nodule) included in the medical image 50X (see FIG. 4) are described in an appropriate description order. The sentence 56X consists of four paragraphs, in order from the beginning of a sentence, each element is described in order so as to be the overall properties of the nodule, the properties of the margins, the internal properties, and the relationship with the surrounding tissue used to determine the degree of progression of the nodule.

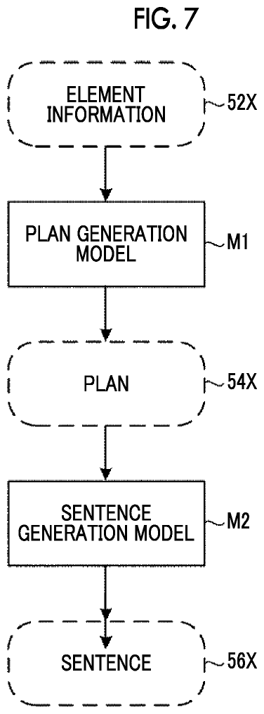

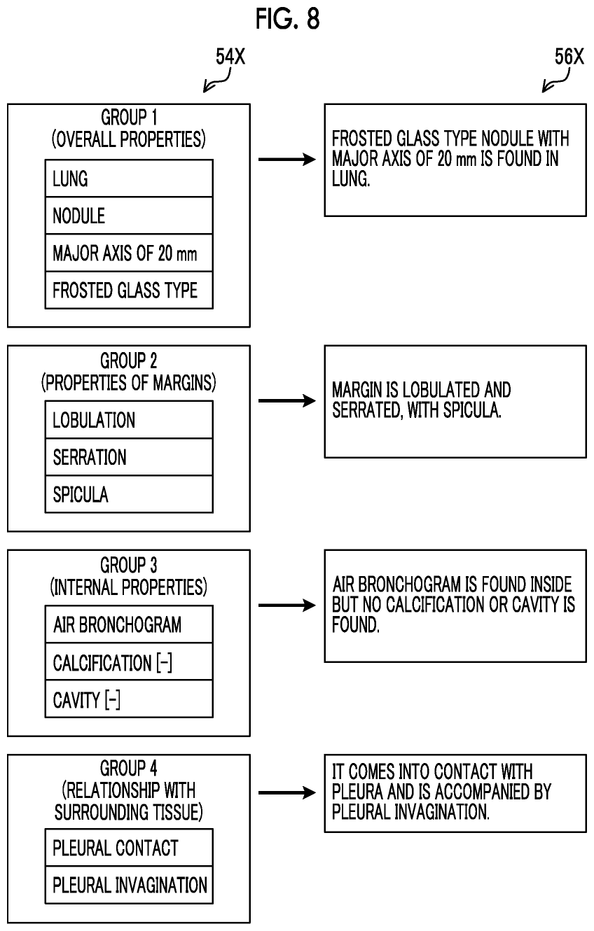

As shown in FIG. 6, the first generation unit 32 and the second generation unit 34 according to the present embodiment generate a sentence in which the elements corresponding to the plurality of pieces of element information acquired and/or generated by the acquisition unit 30 are described in an appropriate description order. Hereinafter, the functions of the first generation unit 32 and the second generation unit 34 will be described with reference to FIGS. 7 and 8. FIG. 7 is a diagram showing the order of processes by the first generation unit 32 and the second generation unit 34 according to the present embodiment.

First, the first generation unit 32 generates a plan which defines the description order of the elements in the sentence in which the elements corresponding to the plurality of pieces of element information acquired and/or generated by the acquisition unit 30 are described. The plan defines the paragraph structure of the entire sentence (that is, the number and order of paragraphs) and in which paragraph each element corresponding to the plurality of pieces of element information is described. The plan defines at least the paragraph structure and in which paragraph each element is described, and the description order of each element in one paragraph may not be defined.

In the generation of the plan, the first generation unit 32 may divide the plurality of pieces of element information acquired and/or generated by the acquisition unit 30 into groups, and generate a plan which defines the description order for each group. That is, one group is regarded as one paragraph, a plurality of pieces of element information are divided into groups, and thus the description order of each paragraph in the entire sentence may be defined by allocating which paragraph each element corresponding to a plurality of pieces of element information is described and defining the order of each group.

FIG. 8 shows a plan 54X as an example of a plan and the sentence 56X generated based on the plan 54X. The plan 54X corresponds to the plurality of pieces of element information 52X (see FIG. 5) related to the medical image 50X (see FIG. 4). In FIG. 8, the plurality of pieces of element information 52X related to the “nodule” are divided into four groups, one showing the overall properties (group 1), one showing the properties of the margins (group 2), one showing the internal properties (group 3), and one showing the relationship with the surrounding tissue (group 4). Groups 1 to 4 correspond to each paragraph arranged in ascending order of number from the beginning of the sentence.

For the generation of the plan by the first generation unit 32, as shown in FIG. 7, the plan generation model M1 such as a CNN and a recurrent neural network (RNN), which has been trained in advance so that the input is the element information and the output is the plan, may be used. The plan generation model M1 is a model that is trained using a set of the element information corresponding to the element included in the sentence generated in the past and the plan which defines the description order of the elements in the sentence as training data. The plan generation model M1 is an example of a first trained model of the present disclosure.

The plan used as the training data of the plan generation model M1 reflects predetermined rules such as the agreement within the medical institution and the user's preference regarding the description order of each element in the sentence as described above. The plan generation model M1 can generate a plan which divides the input element information into groups according to a rule and which defines the description order for each group by learning using such a plan as training data.

Next, the second generation unit 34 generates a sentence based on the plan generated by the first generation unit 32. Specifically, as shown in FIG. 8, the second generation unit 34 generates a paragraph including at least one sentence for each group defined in the plan 54X, and finally collectively generates each paragraph as one sentence 56X.

For the generation of the sentence by the second generation unit 34, as shown in FIG. 7, the sentence generation model M2 such as a CNN and an RNN, which has been trained in advance so that the input is the plan and the output is the sentence, may be used. The sentence generation model M2 is a model that is trained using a set of a sentence generated in the past and a plan related to the sentence as training data. The sentence generation model M2 is an example of a second trained model of the present disclosure.

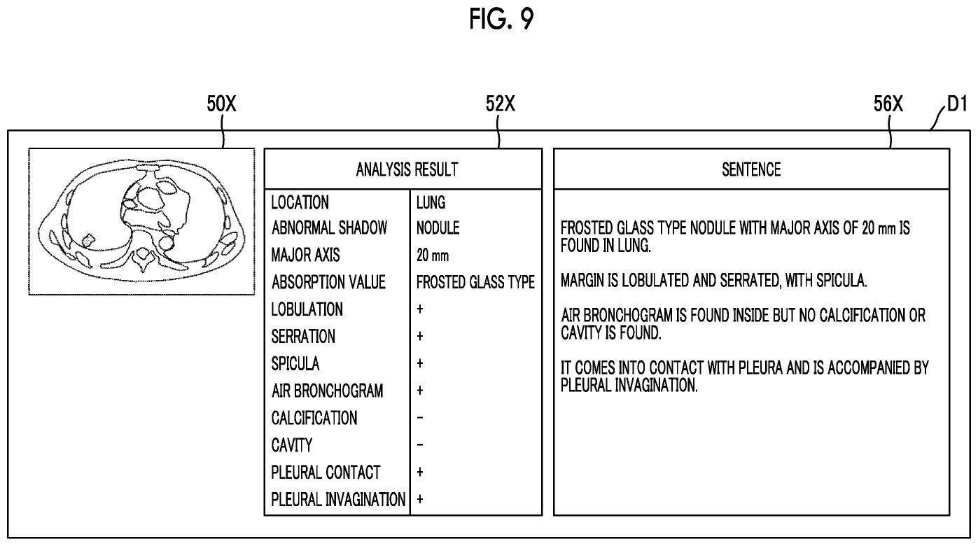

The controller 36 controls the display 24 to display the sentence generated by the second generation unit 34. FIG. 9 shows an example of a screen D1 on which the sentence is displayed, which is displayed on the display 24 by the controller 36. The screen D1 includes the medical image 50X (see FIG. 4), the plurality of pieces of element information 52X acquired and/or generated by the acquisition unit 30 (see FIG. 5), and the sentence 56X generated by the second generation unit 34.

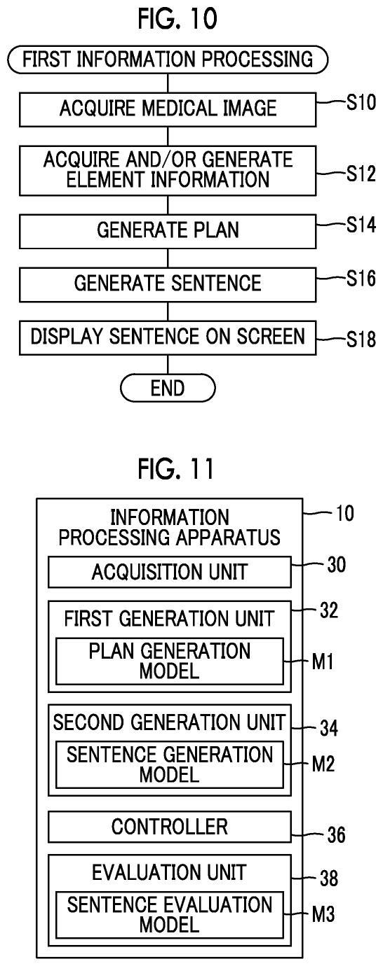

Next, with reference to FIG. 10, operations of the information processing apparatus 10 according to the present embodiment will be described. In the information processing apparatus 10, the CPU 21 executes the information processing program 27, and thus first information processing shown in FIG. 10 is executed. The first information processing is executed, for example, in a case where the user gives an instruction to start execution via the input unit 25.

In Step S10, the acquisition unit 30 acquires a medical image from the image server 5. In Step S12, the acquisition unit 30 acquires and/or generates a plurality of pieces of element information related to the medical image acquired in Step S10. Specifically, the acquisition unit 30 may generate a plurality of pieces of element information based on the medical image acquired in Step S10, or may generate element information based on information input by a user via the input unit 25 and information acquired from an external device. Further, the acquisition unit 30 may acquire element information from an external device.

In Step S14, the first generation unit 32 generates a plan which defines the description order of the elements in the sentence in which the elements corresponding to the plurality of pieces of element information acquired and/or generated in Step S12 are described. In Step S16, the second generation unit 34 generates a sentence based on the plan generated in Step S14. In Step S18, the controller 36 causes the display 24 to display the screen including the sentence generated in Step S16, and ends this first information processing.

As described above, the information processing apparatus 10 according to one aspect of the present disclosure comprises at least one processor, and the processor acquires a plurality of pieces of element information related to an image, generates a plan which defines a description order of elements corresponding to the plurality of pieces of element information in a sentence in which the elements are described, and generates the sentence based on the plan. That is, with the information processing apparatus 10 according to the present embodiment, since it is possible to generate sentences with an appropriate description order and degree of coverage of the elements, it is possible to support the creation of a medical document.

### Second Embodiment

In addition to the same functions as those of the first embodiment, an information processing apparatus 10 according to a second embodiment generates a plurality of plan candidates for a plurality of pieces of element information, evaluates each plan candidate, and selects the most appropriate plan candidate, thereby enabling generation of more appropriate sentences. Hereinafter, the information processing apparatus 10 according to the second embodiment will be described, but the same configurations and functions as those of the first embodiment will be omitted as appropriate.

With reference to FIG. 11, an example of a functional configuration of the information processing apparatus 10 according to the present embodiment will be described. As shown in FIG. 11, the information processing apparatus 10 includes an acquisition unit 30, a first generation unit 32, a second generation unit 34, a controller 36, and an evaluation unit 38. The first generation unit 32 may include a plan generation model M1, and the second generation unit 34 may include a sentence generation model M2. The evaluation unit 38 may include a sentence evaluation model M3 (details will be described later). In a case where the CPU 21 executes the information processing program 27, the CPU 21 functions as the acquisition unit 30, the first generation unit 32, the second generation unit 34, the controller 36, and the evaluation unit 38.

The acquisition unit 30 acquires a medical image to be created as an interpretation report from the image server 5. In addition, the acquisition unit 30 acquires and/or generates a plurality of pieces of element information related to the medical image acquired from the image server 5. Since the function of the acquisition unit 30 is the same as that of the first embodiment, the description thereof will be omitted.

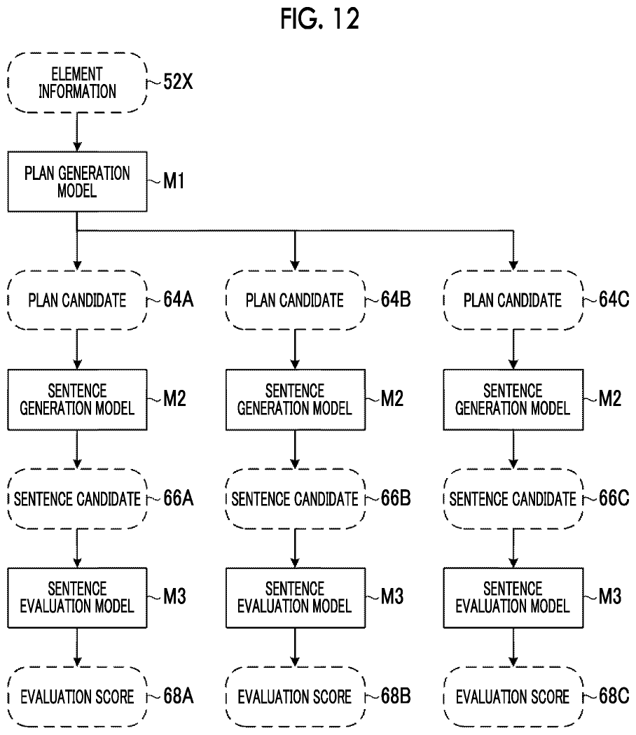

Hereinafter, with reference to FIGS. 12 to 15, the functions of the first generation unit 32, the second generation unit 34, and the evaluation unit 38 according to the present embodiment will be described. FIG. 12 is a diagram showing the order of processes by the first generation unit 32, the second generation unit 34, and the evaluation unit 38 according to the present embodiment. In the following description, a form in which the first generation unit 32, the second generation unit 34, and the evaluation unit 38 generate plan candidates, generate sentences, and evaluate for each paragraph will be described.

First, the first generation unit 32 generates a plurality of different plan candidates for the plurality of pieces of element information acquired and/or generated by the acquisition unit 30. That is, the first generation unit 32 generates a plurality of variations of plan candidates without making the plurality of pieces of element information itself to be generated as a plan candidate different. As a variation of the plan candidates, for example, the number and types of groups in the plan may be different, or the number and types of groups may be the same and which element information is assigned to which group may be different.

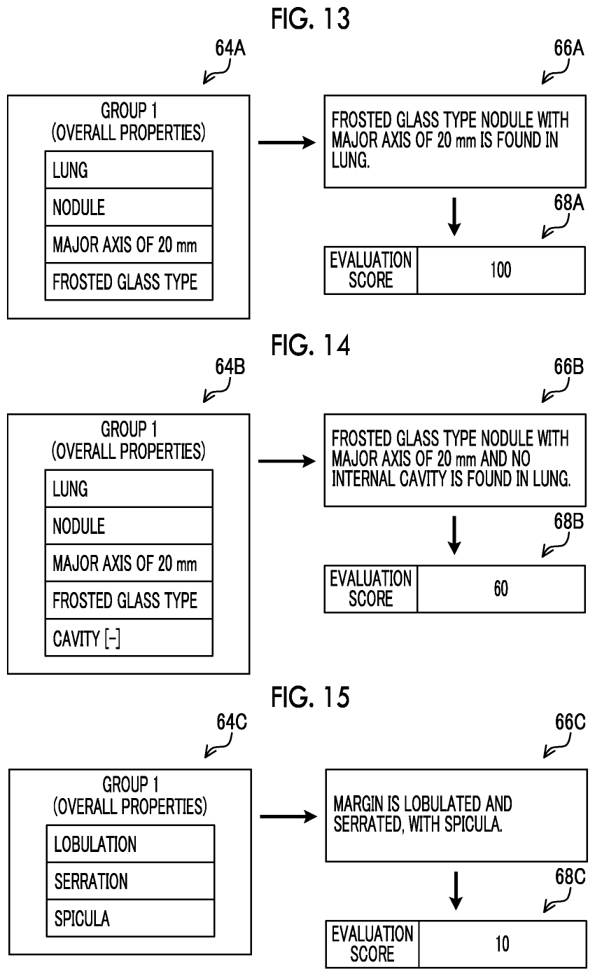

As an example of a plurality of different plan candidates, FIG. 13 shows a plan candidate 64A, FIG. 14 shows a plan candidate 64B, and FIG. 15 shows a plan candidate 64C. Each of the plan candidates 64A to 64C corresponds to the plurality of pieces of element information 52X (see FIG. 5) related to the medical image 50X (see FIG. 4). FIGS. 13 to 15 respectively show, as the plan candidates 64A to 64C, plan candidates for a portion corresponding to one group at the beginning of a sentence (that is, one paragraph at the beginning of a sentence) for a plurality of pieces of element information 52X related to a “nodule”. In this way, the first generation unit 32 first generates a plurality of plan candidates for the portion corresponding to one group (that is, one paragraph).

Since the method of generating the plan candidate by the first generation unit 32 is the same as the method of generating the plan in the first embodiment, the description thereof will be omitted. For example, as shown in FIG. 12, the plan generation model M1 may be used for generation of the plan candidates by the first generation unit 32.

Next, the second generation unit 34 generates a sentence for each plan candidate generated by the first generation unit 32. Specifically, the second generation unit 34 generates sentences for one paragraph (hereinafter referred to as “sentence candidates”) based on the plan candidates for the portion corresponding to one group generated by the first generation unit 32. As an example of sentence candidates generated by the second generation unit 34, sentence candidates 66A to 66C for one paragraph generated based on the plan candidates 64A to 64C for one group are shown in FIGS. 13 to 15, respectively.

Since the method of generating the sentence candidate by the second generation unit 34 is the same as the method of generating the sentence in the first embodiment, the description thereof will be omitted. For example, as shown in FIG. 12, the sentence generation model M2 may be used for the generation of the sentence candidate by the second generation unit 34.

Next, the evaluation unit 38 evaluates each sentence (sentence candidate) generated by the second generation unit 34. Specifically, the evaluation unit 38 performs evaluation based on at least one of the description order or the degree of coverage of the elements included in the sentence (sentence candidate) generated by the second generation unit 34 in the sentence. Evaluation based on the description order means evaluating whether the elements included in the sentence are described in an appropriate order. Evaluation based on the degree of coverage means evaluating whether the elements to be described in the sentence are appropriately described without excess or deficiency. In the present embodiment, since the plan candidates and the sentences (sentence candidates) are generated for each paragraph, the evaluation unit 38 can evaluate the description order and the degree of coverage by evaluating whether the elements to be described in the current evaluation target paragraph are appropriately described without excess or deficiency.

As an example of the result of the evaluation by the evaluation unit 38, evaluation scores 68A to 68C for the sentence candidates 66A to 66C are shown in FIGS. 13 to 15, respectively. The evaluation scores 68A to 68C are scores defined so that the minimum value is 0 and the maximum value is 100, and the better the evaluation, the larger the value. The sentence candidate 66A has the high evaluation score 68A because the elements indicating the overall properties of the nodule are appropriately described. On the other hand, in the sentence candidate 66B, the evaluation score 68B is lower than the evaluation score 68A because the element (“cavity [−]”) indicating the internal properties of the nodule is excessively described. In addition, the sentence candidate 66C has the lowest evaluation score 68C because the elements indicating the properties of margins are described instead of the elements indicating the overall properties of the nodule.

In the evaluation of sentences (sentence candidates) by the evaluation unit 38, as shown in FIG. 12, a sentence evaluation model M3, such as bidirectional encoder representations from transformers (BERT), which has been trained in advance so that the input is a sentence (sentence candidate) and the output is an evaluation score, may be used. The sentence evaluation model M3 is a model that learns sentences generated by a user such as a doctor as correct sentences with an appropriate description order and degree of coverage. That is, the sentence evaluation model M3 is a model for calculating an evaluation score according to the degree of similarity between the input sentence and the pre-learned correct sentence in terms of the description order and the degree of coverage.

In addition, the evaluation unit 38 determines the plan candidate to be employed based on the result of the evaluation of the sentence. For example, in the examples of FIGS. 13 to 15, the plan candidate 64A corresponding to the sentence candidate 66A with the highest evaluation score 68A is selected.

The first generation unit 32, the second generation unit 34, and the evaluation unit 38 repeat the above processing for each paragraph, and determine the plan candidate to be employed for all the paragraphs. This makes it possible to select a plan with an appropriate description order and degree of coverage for each paragraph. In addition, in a case of evaluating a sentence (sentence candidate), the evaluation unit 38 may perform the evaluation in consideration of the evaluation result of the preceding paragraph that has already been evaluated.

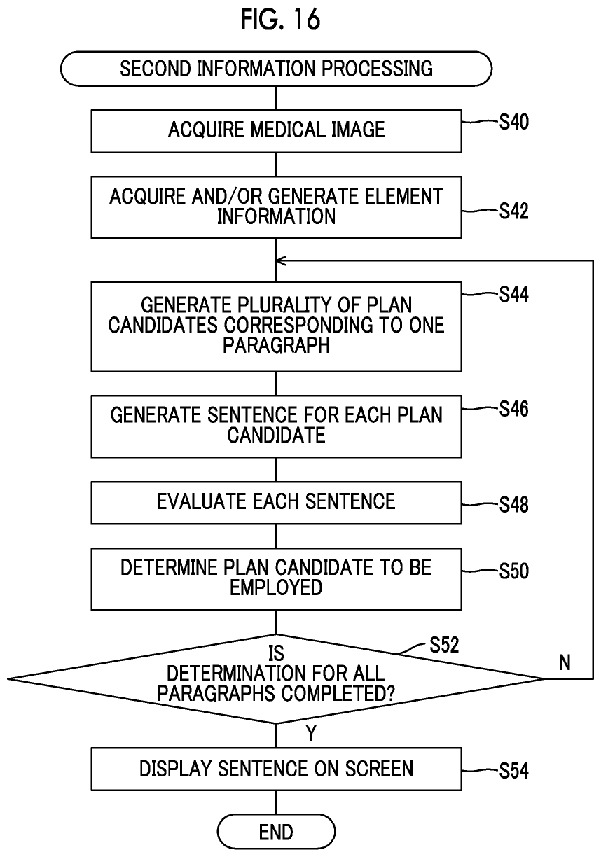

Next, with reference to FIG. 16, operations of the information processing apparatus 10 according to the present embodiment will be described. In the information processing apparatus 10, the CPU 21 executes the information processing program 27, and thus second information processing shown in FIG. 16 is executed. The second information processing is executed, for example, in a case where the user gives an instruction to start execution via the input unit 25.

In Step S40, the acquisition unit 30 acquires a medical image from the image server 5. In Step S42, the acquisition unit 30 acquires and/or generates a plurality of pieces of element information related to the medical image acquired in Step S40. Specifically, the acquisition unit 30 may generate a plurality of pieces of element information based on the medical image acquired in Step S40, or may generate element information based on information input by a user via the input unit 25 and information acquired from an external device. Further, the acquisition unit 30 may acquire element information from an external device.

In Step S44, the first generation unit 32 generates a plurality of plan candidates corresponding to one paragraph of a sentence in which elements corresponding to the plurality of pieces of element information acquired and/or generated in Step S42 are described. In Step S46, the second generation unit 34 generates a sentence (sentence candidate) for each of the plurality of plan candidates generated in Step S44. In Step S48, the evaluation unit 38 evaluates each sentence (sentence candidate) generated in Step S46. In Step S50, the evaluation unit 38 determines a plan candidate to be employed based on the result of evaluation in Step S48.

In Step S52, the evaluation unit 38 determines whether or not the plan candidate to be employed has been determined for all the paragraphs of the sentence in which the elements corresponding to the plurality of pieces of element information acquired and/or generated in Step S42 are described. In a case where the determination of the plan candidate to be employed for all the paragraphs is not completed (that is, in a case where Step S52 is N), the processing of Steps S44 to S50 is repeated for the paragraphs for which the plan candidates have not been generated yet.

On the other hand, in a case where the determination of the plan candidate to be employed for all the paragraphs is completed (that is, in a case where Step S52 is Y), the process proceeds to Step S54. In Step S54, the controller 36 causes the display 24 to display a screen including sentences generated based on the plan candidates for each paragraph determined in Step S50, and ends this second information processing.

As described above, the information processing apparatus 10 according to one aspect of the present disclosure comprises at least one processor, and the processor generates a plurality of different plan candidates for the plurality of pieces of element information, generates the sentence for each plan candidate, evaluates each sentence, and determines the plan candidate to be employed based on the result of the evaluation. That is, with the information processing apparatus 10 according to the present embodiment, since it is possible to generate sentences with a more appropriate description order and degree of coverage, it is possible to support the creation of a medical document.

In the second embodiment, a form in which the evaluation unit 38 determines the plan candidate to be employed among the plurality of plan candidates generated by the first generation unit 32 has been described, but the present disclosure is not limited thereto. For example, the controller 36 may receive the designation of the plan candidate to be employed from among the plurality of different plan candidates generated by the first generation unit 32. Specifically, the controller 36 may control the display 24 to display a plurality of different plan candidates generated by the first generation unit 32, and determine the plan candidate to be employed according to a user's designation via the input unit 25. In this case, the information processing apparatus 10 can omit the function of the evaluation unit 38.

### Comparison With Method of Related Art

A comparison between the methods according to the first and second embodiments and the method of the related art will be described. FIG. 17 is a diagram showing the order of processing for generating sentences from element information by the method of the related art. As shown in FIG. 17, in the method of the related art, the sentence is generated without generating a plan which defines the description order of the elements corresponding to the element information from the element information by using the sentence generation model M0 in the related art, which has been trained in advance so that the input is the element information and the output is the sentence (see, for example, JP2019-153250A).

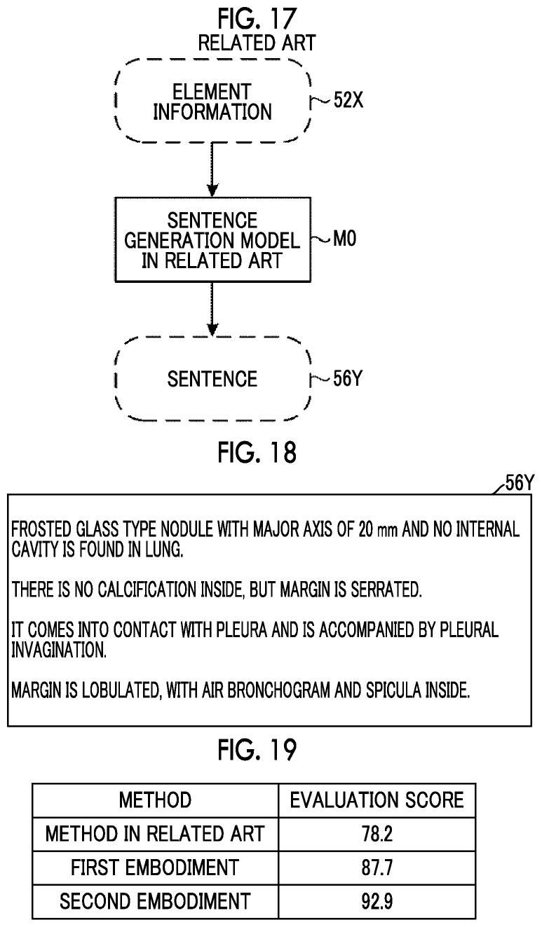

In the method using the sentence generation model M0 in the related art, in a case where the element information becomes large and complicated, there are cases where the generated sentences are not in the desired order of description, the information is omitted, or the sentences become redundant. That is, there are cases where the description order and the degree of coverage of the sentences become inappropriate. FIG. 18 shows an example of a sentence 56Y obtained by inputting the plurality of pieces of element information 52X (see FIG. 5) related to the medical image 50X (see FIG. 4) into the sentence generation model MO in the related art. Compared with the sentence 56X shown in FIG. 6, the sentence 56Y has an inappropriate description order and is difficult for the reader to read.

FIG. 19 shows the results of evaluating each of the sentences generated by the methods according to the first and second embodiments of the present disclosure and the sentences generated by the method of the related art. FIG. 19 shows an “evaluation score” representing the appropriateness of the description order and the degree of coverage of the elements in the sentence, which is calculated by the sentence evaluation model M3. As shown in FIG. 19, it can be seen that the sentences generated by the methods according to the first and second embodiments of the present disclosure have a higher evaluation score than the sentences generated by the method of the related art, and the appropriateness of the description order and the degree of coverage of the elements in the sentence is improved.

In each of the above embodiments, a form for generating a plan and a sentence for the plurality of pieces of element information 52X related to one region of interest (abnormal shadow N) included in one medical image 50X has been described, but the present disclosure is not limited thereto.

For example, the acquisition unit 30 may acquire and/or generate a plurality of pieces of element information related to each of a plurality of regions of interest included in one image, and the first generation unit 32 may generate a plan by dividing the plurality of pieces of element information into a plurality of groups corresponding to each of the plurality of regions of interest. The second generation unit 34 may generate a sentence in which the elements corresponding to the plurality of pieces of element information related to each of the plurality of regions of interest are collectively described.

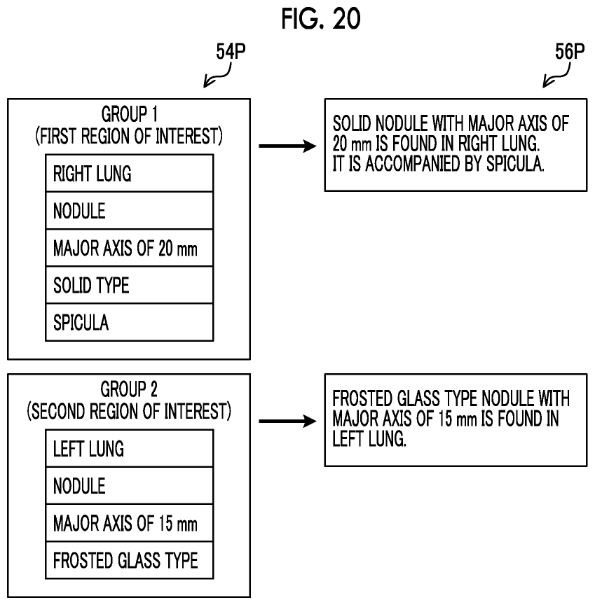

FIG. 20 shows a plan 54P assuming a case where abnormal shadows showing nodules are included in each of the right lung and the left lung for the medical image obtained by imaging the lungs, and a sentence 56P generated based on the plan 54P. That is, FIG. 20 assumes a case where one medical image including both the right lung and the left lung includes a plurality of abnormal shadows. In the plan 54P, the groups are divided into element information related to nodules included in the right lung (group 1) and element information related to nodules included in the left lung (group 2). In this way, the first generation unit 32 may generate a plan by dividing the plurality of pieces of element information into a plurality of groups corresponding to each of the plurality of abnormal shadows.

Further, for example, the acquisition unit 30 may acquire and/or generate a plurality of pieces of element information related to each of regions of interest included in a plurality of images, and the first generation unit 32 may generate a plan by dividing the plurality of pieces of element information into a plurality of groups corresponding to each of the plurality of images. That is, the plurality of pieces of element information acquired and/or generated by the acquisition unit 30 may be related to each of the plurality of images. The second generation unit 34 may generate a sentence in which the elements corresponding to the plurality of pieces of element information related to each of the plurality of images are collectively described.

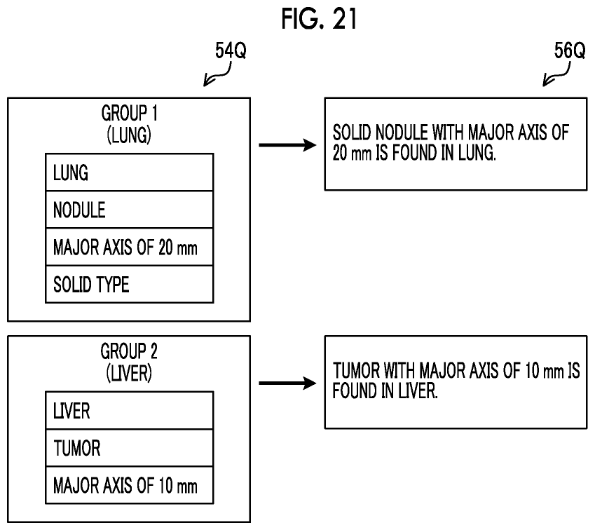

FIG. 21 shows a plan 54Q assuming a case where abnormal shadows showing nodules are included in each of a medical image in which the lung part is imaged and a medical image in which the liver part is imaged for the same subject, and a sentence 56Q generated based on the plan 54Q. That is, FIG. 21 assumes a case where the medical image of the lung and the medical image of the liver are different images. In the plan 54Q, the groups are divided into element information related to nodules included in the lungs (group 1) and element information related to nodules included in the liver (group 2). In this way, the first generation unit 32 may generate a plan by dividing a plurality of pieces of element information into a plurality of groups corresponding to each of a plurality of images.

Further, for example, the acquisition unit 30 may acquire and/or generate element information indicating an imaging point in time of the image, and the first generation unit 32 may generate the plan which defines the description order of the elements corresponding to the related element information based on the imaging point in time indicated by the element information. The “imaging point in time” may represent, for example, the date and time when the imaging was performed, or may represent the imaging phase such as the arterial phase, portal vein phase, and equilibrium phase in the contrast medium examination. In this case, the first generation unit 32 may generate a plan by dividing a plurality of pieces of element information into a plurality of groups corresponding to each of a plurality of imaging points in time. The second generation unit 34 may generate a sentence in which the elements corresponding to the plurality of pieces of element information related to each of the plurality of images captured at a plurality of different imaging points in time are collectively described.

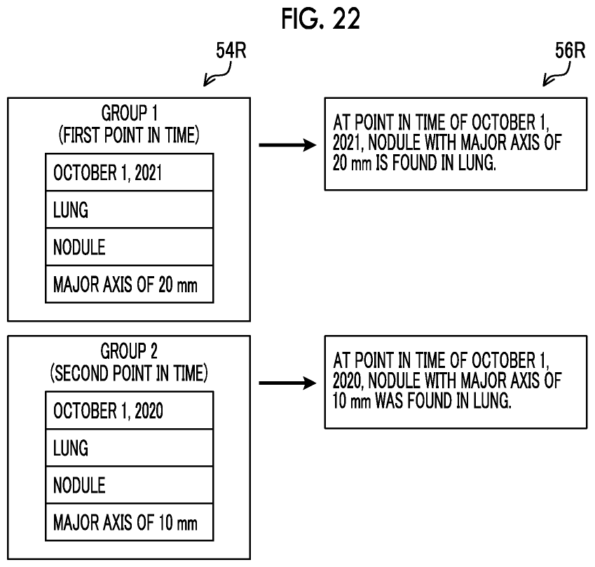

FIG. 22 shows a plan 54R assuming a case where abnormal shadows showing nodules are included in each of the medical images obtained by imaging the lungs of the same subject at different points in time (dates and times), and a sentence 56R generated based on the plan 54R. In the plan 54R, the groups are divided into element information related to nodules included in the medical image captured at the first point in time (group 1) and element information related to nodules included in the medical image captured at the second point in time (group 2). In this way, the first generation unit 32 may generate a plan by dividing a plurality of pieces of element information into a plurality of groups corresponding to each of a plurality of imaging points in time.

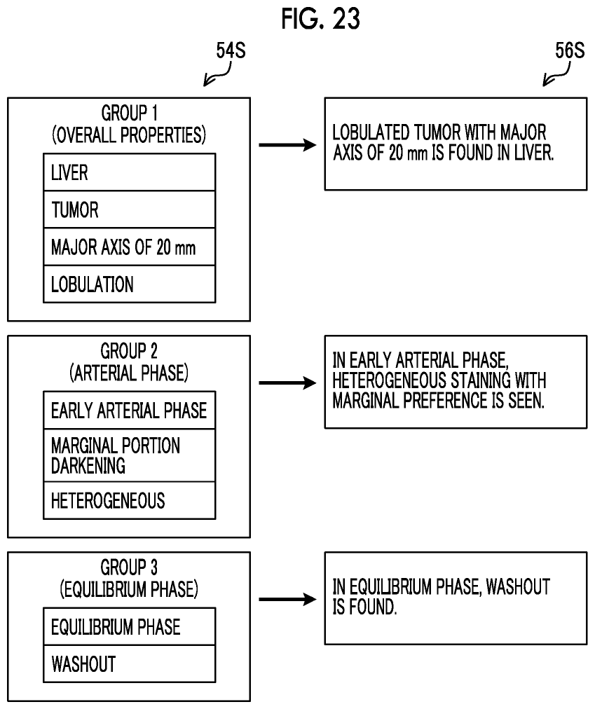

Further, FIG. 23 shows a plan 54S assuming a case where abnormal shadows are included in each of the medical images of each phase obtained by imaging the liver with a contrast medium for the same subject, and a sentence 56S generated based on the plan 54S. In the plan 54S, the groups are divided into element information related to the overall properties common to each phase (group 1), element information related to the arterial phase (group 2), and element information related to the equilibrium phase (group 3). In this way, the first generation unit 32 may generate a plan by dividing the plurality of pieces of element information into a plurality of groups corresponding to each of a plurality of imaging phases.

Further, for example, in a case where the first generation unit 32 generates a plan for element information related to each of a plurality of regions of interest, the first generation unit may derive the degree of severity for each region of interest, and generate a plan such that elements corresponding to element information related to the region of interest with a higher degree of severity are described closer to the beginning of the sentence. The degree of severity of the region of interest can be derived, for example, from the size, position, absorption value, and the like of the region of interest. In the example of FIG. 20, the first generation unit 32 may generate a plan such that an element related to a first region of interest, which is relatively large and solid and has spicula, is described at the beginning of the sentence, and an element related to a second region of interest, which is relatively small and of a frosted glass type, is described at the end of the sentence.

Further, for example, the first generation unit 32 may generate a plan such that, in a case where a plurality of regions of interest can be estimated to be the metastasis source and metastasis destination of tumor cells, respectively, the elements related to the metastasis source are described at the beginning of the sentence and the elements related to the metastasis destination are described at the end of the sentence. The metastasis source and metastasis destination can be estimated from, for example, the size, position, absorption value, and the like of the region of interest. In addition, in a case where the same subject is imaged at different points in time (dates and times), it can be estimated from the point in time at which abnormal shadows are detected. In the example of FIG. 21, the first generation unit 32 may generate a plan such that a relatively large lung nodule is estimated to be the metastasis source and a relatively small liver tumor is estimated to be the metastasis destination, and elements related to lungs are described at the beginning of the sentence and elements related to the liver are described at the end of the sentence.

Further, in each of the above embodiments, an example in which the first generation unit 32 generates a plan including all of the plurality of pieces of element information acquired and/or generated by the acquisition unit 30 has been described, but the present disclosure is not limited thereto. In a case where there are a large number of a plurality of pieces of element information, the first generation unit 32 may generate a plan by selecting the plurality of pieces of element information acquired and/or generated by the acquisition unit 30 in order to suppress the lengthening of the sentence. In this case, the importance may be predetermined for each piece of element information and may be stored in advance in the storage unit 22 or the like. For example, with respect to nodules, the importance of the element information corresponding to the element that can be the basis of malignancy (for example, spicula) may be increased, and the importance of the element information corresponding to the element that can be the basis of benign (for example, clear boundary) may be decreased.

Specifically, the first generation unit 32 may generate a plan which defines that an element corresponding to element information having a relatively low importance among the plurality of pieces of element information acquired and/or generated by the acquisition unit 30 is not described in the sentence. For example, in a case where the number of a plurality of pieces of element information acquired and/or generated by the acquisition unit 30 exceeds a predetermined threshold value, the first generation unit may generate a plan by selecting the plurality of pieces of element information such that the number of pieces of element information exceeding the threshold value are not described in the sentence in descending order of importance.

Further, the first generation unit 32 may generate the plan which defines that an element corresponding to element information whose importance is lower than a predetermined threshold value among the plurality of pieces of element information acquired and/or generated by the acquisition unit 30 is not described in the sentence. Here, the predetermined threshold value may be optionally set by the user. That is, regardless of the number of the plurality of pieces of element information acquired and/or generated by the acquisition unit 30, the elements corresponding to the element information whose importance is lower than the predetermined importance may not be described in the sentence.

Further, the first generation unit 32 may generate a plan such that the elements corresponding to the element information having a higher importance are described at the beginning of the sentence.

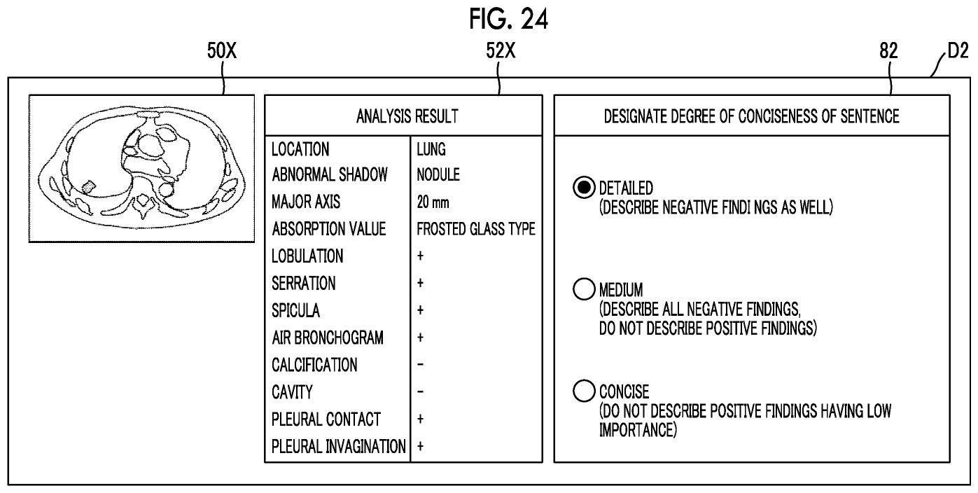

Further, the first generation unit 32 may receive a designation of a degree of conciseness of the sentence generated by the second generation unit 34, and change the number of elements not to be described in the sentence depending on the designated degree of conciseness. FIG. 24 shows an example of a screen D2 on which a designation of the degree of conciseness of the sentence is received, which is displayed on the display 24 by the controller 36. The screen D2 includes the medical image 50X (see FIG. 4), the plurality of pieces of element information 52X acquired and/or generated by the acquisition unit 30 (see FIG. 5), and a designation field 82 for receiving a designation of the degree of conciseness of the sentence. In this case, the user designates one of the degrees of conciseness of the sentences displayed in the designation field 82 on the screen D2 displayed on the display 24 via the input unit 25. The first generation unit 32 generates a plan based on the plurality of pieces of element information 52X according to the degree of conciseness of the sentence designated by the user.

In addition, for example, an element indicating the presence or absence of calcification of a nodule is usually desired to be described in a sentence because it can be a basis for determining whether the nodule is benign or malignant. On the other hand, in a case where the nodule is of the frosted glass type, calcification is generally not observed, and thus some users prefer to omit the description. Therefore, the first generation unit 32 may change whether or not to describe an element corresponding to another piece of element information in a sentence, based on the presence or absence and degree of certain element information, such as lowering the importance of element information indicating “calcification” in a case where there is element information indicating a “frosted glass type”, for example.

As described above, the information processing apparatus 10 according to each of the above embodiments can generate various plans according to a plurality of different predetermined rules, such as for each region (the overall properties, the properties of margins, and the like) related to one region of interest, for each of a plurality of regions of interest, for each of a plurality of images, and for each of a plurality of imaging points in time. Therefore, the first generation unit 32 may include a plurality of plan generation models for generating various plans according to different rules. For example, the first generation unit 32 may include four plan generation models, that is, a model for generating a plan in which groups are divided by regions related to one region of interest, a model for generating a plan in which groups are divided by a plurality of regions of interest, a model for generating a plan in which groups are divided by a plurality of images, and a model for generating a plan in which groups are divided by a plurality of imaging points in time.

Each of the plurality of plan generation models is a model such as a CNN and an RNN, which has been trained in advance so that the input is the element information and the output is the plan. Each of the plurality of plan generation models is a model that is trained using a set of the element information corresponding to the element included in the sentence generated in the past and the plan which defines the description order of the elements in the sentence as training data, but the training data differs depending on the model. For example, as a model for generating a plan in which groups are divided by regions related to one region of interest, a plan in which groups are divided by regions related to one region of interest is used as training data. On the other hand, as a model for generating a plan in which groups are divided by a plurality of regions of interest, a plan in which groups are divided by a plurality of regions of interest is used as training data.

The first generation unit 32 may selectively use at least one of a plurality of plan generation models to generate a plan. Regarding which plan generation model to select, for example, the first generation unit 32 may select the optimum plan generation model based on a plurality of pieces of element information acquired and/or generated by the acquisition unit 30.

Further, for example, the first generation unit 32 may receive a designation of at least one of a plurality of different predetermined rules into the groups, and divide the plurality of pieces of element information into groups according to the designated rule. Specifically, the first generation unit 32 may receive the user's designation as to which plan generation model to select from the plurality of plan generation models.

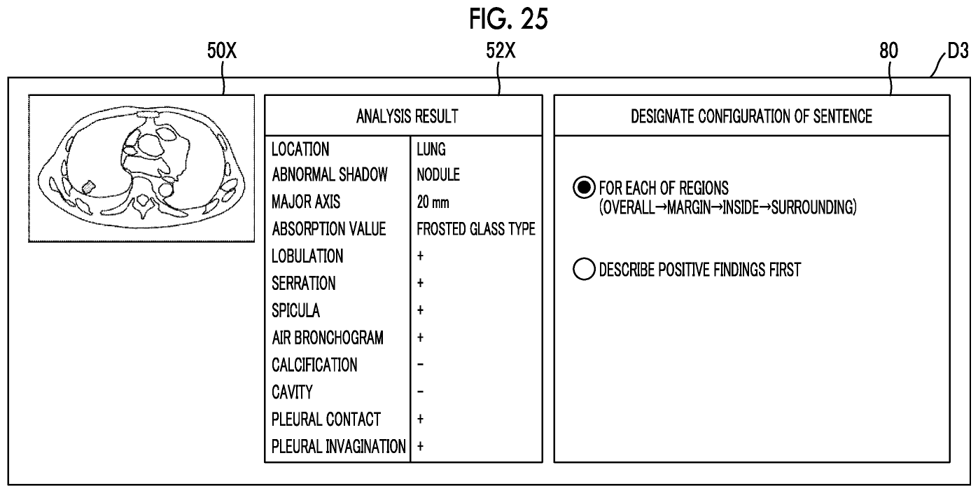

FIG. 25 shows an example of a screen D3 on which a designation of the plurality of different rules is received, which is displayed on the display 24 by the controller 36. The screen D3 includes the medical image 50X (see FIG. 4), the plurality of pieces of element information 52X acquired and/or generated by the acquisition unit 30 (see FIG. 5), and a designation field 80 for receiving a designation of the plurality of different rules. In this case, the user designates at least one of the plurality of different rules displayed in the designation field 80 on the screen D3 displayed on the display 24 via the input unit 25.

The first generation unit 32 divides the plurality of pieces of element information 52X into groups according to at least one rule designated by the user. Specifically, the first generation unit 32 may selectively use a plan generation model trained in advance using training data according to at least one rule designated by the user among the plurality of plan generation models to generate a plan.

Further, in each of the above embodiments, the form in which the medical image is used as an example of the image has been described, but the technique of the present disclosure can also use an image other than the medical image. For example, the technique of the present disclosure can also be applied in a case of creating a report on images (for example, CT images, visible light images, infrared images, and the like) captured in non-destructive inspection of civil engineering structures, industrial products, pipes, and the like. In this case, as the element information related to the image, information indicating at least one of a name, a property, a measured value, or a position related to a region of interest included in the image, or an imaging method, an imaging condition, or an imaging date and time related to imaging of the image can be applied.

In the above embodiments, for example, as hardware structures of processing units that execute various kinds of processing, such as the acquisition unit 30, the first generation unit 32, the second generation unit 34, the controller 36, and the evaluation unit 38, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (program).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example in which a plurality of processing units are configured by one processor, first, there is a form in which one processor is configured by a combination of one or more CPUs and software as typified by a computer, such as a client or a server, and this processor functions as a plurality of processing units. Second, as represented by a system on chip (SoC) or the like, there is a form of using a processor for realizing the function of the entire system including a plurality of processing units with one integrated circuit (IC) chip. In this way, various processing units are configured by one or more of the above-described various processors as hardware structures.

Furthermore, as the hardware structure of the various processors, more specifically, an electrical circuit (circuitry) in which circuit elements such as semiconductor elements are combined can be used.

In the above embodiment, the information processing program 27 is described as being stored (installed) in the storage unit 22 in advance; however, the present disclosure is not limited thereto. The information processing program 27 may be provided in a form recorded in a recording medium such as a compact disc read only memory (CD-ROM), a digital versatile disc read only memory (DVD-ROM), and a universal serial bus (USB) memory. In addition, the information processing program 27 may be downloaded from an external device via a network. Further, the technique of the present disclosure extends to a storage medium for storing the information processing program non-transitorily in addition to the information processing program.

The technique of the present disclosure can be appropriately combined with the above-described embodiments. The described contents and illustrated contents shown above are detailed descriptions of the parts related to the technique of the present disclosure, and are merely an example of the technique of the present disclosure. For example, the above description of the configuration, function, operation, and effect is an example of the configuration, function, operation, and effect of the parts according to the technique of the present disclosure. Therefore, needless to say, unnecessary parts may be deleted, new elements may be added, or replacements may be made to the described contents and illustrated contents shown above within a range that does not deviate from the gist of the technique of the present disclosure.

What is claimed is:

1. An information processing apparatus comprising at least one processor, wherein the at least one processor is configured to:
acquire a plurality of pieces of element information related to an image;
generate a plan which defines a description order of elements corresponding to the plurality of pieces of element information in a sentence in which the elements are described; and
generate the sentence based on the plan.

2. The information processing apparatus according to claim 1, wherein the at least one processor is configured to:
divide the plurality of pieces of element information into groups; and
generate the plan which defines the description order for each group.

3. The information processing apparatus according to claim 2, wherein the at least one processor is configured to:
receive a designation of at least one of a plurality of different rules predetermined for a method of dividing the plurality of pieces of element information into the groups; and
divide the plurality of pieces of element information into groups according to the designated rule.

4. The information processing apparatus according to claim 2, wherein the at least one processor is configured to:
acquire a plurality of pieces of element information related to each of a plurality of regions of interest included in the image; and
divide the plurality of pieces of element information into a plurality of groups corresponding to each of the plurality of regions of interest.

5. The information processing apparatus according to claim 1, wherein:
the plurality of pieces of element information are related to each of a plurality of the images, and
the at least one processor is configured to generate a sentence in which elements corresponding to the plurality of pieces of element information related to each of the plurality of images are collectively described.

6. The information processing apparatus according to claim 1, wherein the at least one processor is configured to:
acquire element information indicating an imaging point in time of the image; and
generate the plan which defines the description order of an element corresponding to related element information based on the imaging point in time indicated by the element information.

7. The information processing apparatus according to claim 1, wherein:
an importance is predetermined for each piece of element information, and
the at least one processor is configured to generate the plan which defines that an element corresponding to element information whose importance is lower than a predetermined threshold value among the plurality of pieces of element information is not described in the sentence.

8. The information processing apparatus according to claim 1, wherein:
an importance is predetermined for each piece of element information, and
the at least one processor is configured to generate the plan which defines that an element corresponding to element information having a relatively low importance among the plurality of pieces of element information is not described in the sentence.

9. The information processing apparatus according to claim 7, wherein the at least one processor is configured to:
receive a designation of a degree of conciseness of the sentence; and
change the number of elements not to be described in the sentence depending on the degree of conciseness.

10. The information processing apparatus according to claim 1, wherein the at least one processor is configured to:
generate the plan using a first trained model that has been trained in advance so that an input is the element information and an output is the plan; and
generate the sentence using a second trained model that has been trained in advance so that an input is the plan and an output is the sentence.

11. The information processing apparatus according to claim 10, wherein the first trained model is trained using a set of the element information corresponding to the element included in the sentence generated in a past and the plan which defines the description order of the elements in the sentence as training data.

12. The information processing apparatus according to claim 1, wherein the at least one processor is configured to:
generate a plurality of different candidates of the plan for the plurality of pieces of element information;
generate the sentence for each candidate of the plan;
evaluate each sentence; and
determine a candidate of the plan to be employed based on a result of the evaluation.

13. The information processing apparatus according to claim 12, wherein the at least one processor is configured to perform the evaluation based on at least one of the description order or a degree of coverage of the elements included in the generated sentence in the sentence.

14. The information processing apparatus according to claim 1, wherein the at least one processor is configured to:
generate a plurality of different candidates of the plan for the plurality of pieces of element information; and
receive a designation of a candidate of the plan to be employed from among the plurality of different candidates of the plan.

15. The information processing apparatus according to claim 1, wherein the at least one processor is configured to:
acquire the image; and
generate the element information based on the acquired image.

16. The information processing apparatus according to claim 1, further comprising an input unit,
wherein the at least one processor is configured to generate the element information based on information input via the input unit.

17. The information processing apparatus according to claim 1, wherein the element information is information indicating at least one of a name, a property, a measured value, or a position related to a region of interest included in the image, or an imaging method, an imaging condition, or an imaging date and time related to imaging of the image.

18. The information processing apparatus according to claim 1, wherein:
the image is a medical image,
the element information is information indicating at least one of a name, a property, a position, or an estimated disease name related to a region of interest included in the medical image, or an imaging method, an imaging condition, or an imaging date and time related to imaging of the medical image, and
the region of interest is at least one of a region of a structure included in the medical image or a region of an abnormal shadow included in the medical image.

19. An information processing method comprising:
acquiring a plurality of pieces of element information related to an image;
generating a plan which defines a description order of elements corresponding to the plurality of pieces of element information in a sentence in which the elements are described; and
generating the sentence based on the plan.

20. A non-transitory computer-readable storage medium storing an information processing program causing a computer to execute:
acquiring a plurality of pieces of element information related to an image;
generating a plan which defines a description order of elements corresponding to the plurality of pieces of element information in a sentence in which the elements are described; and
generating the sentence based on the plan.
